(19) United States
(12) Patent Application Publication
(10) Pub. No.: US 2024/0177824 A1
(43) Pub. Date: May 30, 2024

(54) MONITORING FOOD CONSUMPTION USING AN ULTRAWIDE BAND SYSTEM

(71) Applicant: Meta Platforms Technologies, LLC, Menlo Park, CA (US)

(72) Inventors: Sophia Hyein Kim, Sunnyvale, CA (US); Nabeel Ahmed, San Jose, CA (US)

(21) Appl. No.: 17/940,807

(22) Filed: Sep. 8, 2022

Related U.S. Application Data
(60) Provisional application No. 63/247,402, filed on Sep. 23, 2021.

(51) Int. Cl. G16H 20/60; A61B 5/00; A61B 5/11; G01S 13/02; G06V 20/68

(52) U.S. Cl. CPC G16H 20/60; A61B 5/1123; A61B 5/6803; G01S 13/0209; G06V 20/68

(57) ABSTRACT

A system, a headset, or a method for determining a value of a food consumption parameter. The system includes a headset worn on a head of a user and a wearable device worn on a wrist or a hand of the user. The headset and the wearable device are communicatively coupled to each other via an ultrawideband communication channel. The system tracks the hand of the user relative to the head of the user based on the ultrawideband communication channel between the headset and the wearable device. The system also monitors movement of a jaw of the user using a contact microphone coupled to the headset, and determines a value of a food consumption parameter based in part on the tracked movement of the hand and the monitored movement of the jaw.

### CROSS-REFERENCE TO RELATED APPLICATIONS

This application claims the benefit of U.S. Provisional Application No. 63/247,402, filed Sep. 23, 2021, which is incorporated by reference in its entirety.

### FIELD OF THE INVENTION

This disclosure relates generally to monitoring food consumption, and more specifically to monitoring food consumption using an ultrawideband system.

### BACKGROUND

Conventionally food consumption is monitored by a person individually tracking and logging what they eat, how much they eat, when they eat, etc. The log may be hand written or input into an application that simply provides calorie estimates for the portions and types of foods input. Accuracy of both instances rely heavily on how diligent the person is in tracking what food was consumed. As such, utility of conventional systems goes down for people who do not have the time/dedication to accurately track what food was consumed. Moreover, even if a person accurately tracks what food was consumed, conventional systems are not able to actively monitor, e.g., whether a user is choking and/or whether a user is having a hard time eating (e.g., having a hard time picking up their food, etc.).

### SUMMARY

Embodiments relate to monitoring food consumption using an ultrawideband (UWB) system. The UWB system includes a headset and at least one wearable device (e.g., bracelet, watch, ring) that couples to a wrist or a hand and/or hand of a user. The headset and the at least one wearable device communicate with each other via a UWB connection. The headset includes a contact microphone that measures skin vibrations. The UWB system monitors signals from the contact microphone and the position of the hand relative to the mouth in order to determine values of food consumption parameters. In some embodiments, determining the values of food consumption parameters is performed by the headset. A food consumption parameter describes aspects (e.g., calories, choking, food handling, etc.) of food consumption. The UWB system may present the determined values to the user (e.g., via display on the headset, an application on a smartphone, etc.).

Embodiments also relate to a method for monitoring food consumption. The method includes tracking movement of a hand of a user relative to a head of the user based on an ultrawideband communication channel between a headset worn by the user and a wearable device worn on the hand or a corresponding wrist of the user. The method further includes monitoring movement of a jaw of the target user using a contact microphone coupled to the headset, and determining a value of a food consumption parameter of the user based in part on the tracked movement of the hand and the monitored movement of the jaw.

### BRIEF DESCRIPTION OF THE DRAWINGS

FIG. 1A is a perspective view of a headset implemented as an eyewear device, in accordance with one or more embodiments.

FIG. 1B is a perspective view of a headset implemented as a head-mounted display, in accordance with one or more embodiments.

FIG. 2 is a block diagram of a UWB system, in accordance with one or more embodiments.

FIG. 3 illustrates an example use case of the UWB system of FIG. 2, where the UWB system is worn by a user for track values of food consumption parameters of the user.

FIG. 4 is a flowchart of a method for determining a value of a food consumption parameter, in accordance with one or more embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

Embodiments relate to monitoring food consumption using an ultrawideband (UWB) system. The UWB system includes a headset and at least one wearable device (e.g., bracelet, watch, ring) that couples to a wrist or a hand and/or hand of a user. In some embodiments, the UWB system may also include some other device (e.g. smartphone). The headset and the at least one wearable device communicate with each other via a UWB connection. UWB protocol is a wireless communication protocol that uses radio waves (or pulses) to transmit data and precisely determine locations of devices. For example, the UWB connection may be used to determine a position of the wearable device (i.e., the hand) relative to the headset (e.g., the mouth of the user).

Traditionally, distances between two devices may be measured based on the strength of a narrow band signal, such as Bluetooth Low Energy (BLE) signals or Wi-Fi signals. Such traditional communication protocols provide an accuracy of several meters. Unlike the traditional communication protocols, UWB uses short bursts of signals with sharp rises and drops. The start and stop times of the UWB signals can be accurately measured, which can then be used to precisely measure a distance between two UWB devices. In particular, UWB can provide an accuracy of a few centimeters. As such, one advantage to UWB over traditional communication protocols is that it can be used to more accurately determine positional information between two devices. Additionally, since UWB uses short bursts of signals to communicate, it also consumes less energy compared to the traditional communication protocols.

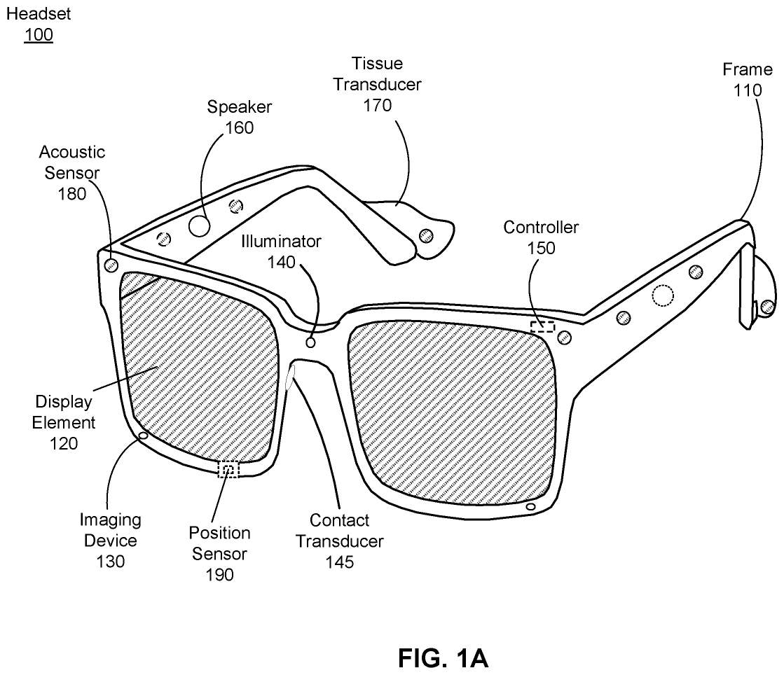

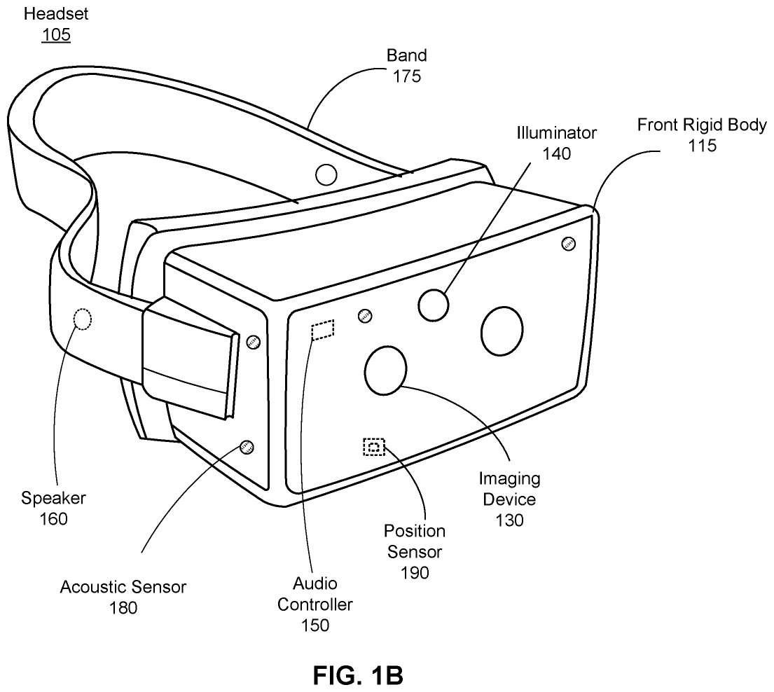

The headset includes a contact microphone that measures skin vibrations. The UWB system monitors signals from the contact microphone and the position of the hand relative to the mouth in order to determine values of food consumption parameters. A food consumption parameter describes aspects (e.g., calories, choking, food handling, etc.) of food consumption. The UWB system may present the determined values to the user (e.g., via display on the headset, an application on a smartphone, etc.). The headset is communicatively coupled with one or more devices. An example headset is described below with regard to FIG. 1A or 1B. One advantage to UWB over traditional communication protocols is that it is not only able to transmit communication data between multiple devices, but also can be used to determine positional information of the multiple devices. For example, the UWB connection may be used to determine a position of the wearable device (i.e., the hand) relative to the headset (e.g., the mouth of the user).

In some embodiments, short bursts of signals are transmitted between the headset and the wearable device over the UWB channel. The signals with sharp rises and drops make it easier to measure. The distance between the headset and the wearable device can be measured precisely by measuring the time that it takes for a radio wave to pass between the two devices, which delivers a much more precise distance measurement than measurement based on signal strength, such as using the strength of BLE signals. It is advantageous to use UWB compared to other narrowband radio systems, such as Bluetooth low energy (BLE) or Wi-Fi, to detect relative positions between the headset and the wearable device, because UWB uses less energy and can measure distance and location with an accuracy of 5 to 10 centimeters, while Wi-Fi, BLE, and other narrowband radio systems generally can only reach an accuracy of several meters.

Embodiments of the invention may include or be implemented in conjunction with an artificial reality system. Artificial reality is a form of reality that has been adjusted in some manner before presentation to a user, which may include, e.g., a virtual reality (VR), an augmented reality (AR), a mixed reality (MR), a hybrid reality, or some combination and/or derivatives thereof. Artificial reality content may include completely generated content or generated content combined with captured (e.g., real-world) content. The artificial reality content may include video, audio, haptic feedback, or some combination thereof, any of which may be presented in a single channel or in multiple channels (such as stereo video that produces a three-dimensional effect to the viewer). Additionally, in some embodiments, artificial reality may also be associated with applications, products, accessories, services, or some combination thereof, that are used to create content in an artificial reality and/or are otherwise used in an artificial reality. The artificial reality system that provides the artificial reality content may be implemented on various platforms, including a wearable device (e.g., headset) connected to a host computer system, a standalone wearable device (e.g., headset, a bracelet, a watch, a ring), a mobile device or computing system, or any other hardware platform capable of providing artificial reality content to one or more viewers.

FIG. 1A is a perspective view of a headset 100 implemented as an eyewear device, in accordance with one or more embodiments. The headset 100 is an example of the headset described above in communication (e.g., via UWB) with one or more wearable devices. In some embodiments, the eyewear device is a near eye display (NED). In general, the headset 100 may be worn on the face of a user such that content (e.g., media content) is presented using a display assembly and/or an audio system. However, the headset 100 may also be used such that media content is presented to a user in a different manner. Examples of media content presented by the headset 100 include one or more images, video, audio, or some combination thereof. The headset 100 includes a frame, and may include, among other components, a display assembly including one or more display elements 120, a depth camera assembly (DCA), an audio system, a controller 150, and a position sensor 190. While FIG. 1A illustrates the components of the headset 100 in example locations on the headset 100, the components may be located elsewhere on the headset 100, on a peripheral device paired with the headset 100, or some combination thereof. Similarly, there may be more or fewer components on the headset 100 than what is shown in FIG. 1A.

The frame 110 holds the other components of the headset 100. The frame 110 includes a front part that holds the one or more display elements 120 and end pieces (e.g., temples) to attach to a head of the user. The front part of the frame 110 bridges the top of a nose of the user. The length of the end pieces may be adjustable (e.g., adjustable temple length) to fit different users. The end pieces may also include a portion that curls behind the ear of the user (e.g., temple tip, ear piece).

The one or more display elements 120 provide light to a user wearing the headset 100. As illustrated the headset includes a display element 120 for each eye of a user. In some embodiments, a display element 120 generates image light that is provided to an eyebox of the headset 100. The eyebox is a location in space that an eye of user occupies while wearing the headset 100. For example, a display element 120 may be a waveguide display. A waveguide display includes a light source (e.g., a two-dimensional source, one or more line sources, one or more point sources, etc.) and one or more waveguides. Light from the light source is in-coupled into the one or more waveguides which outputs the light in a manner such that there is pupil replication in an eyebox of the headset 100. In-coupling and/or outcoupling of light from the one or more waveguides may be done using one or more diffraction gratings. In some embodiments, the waveguide display includes a scanning element (e.g., waveguide, mirror, etc.) that scans light from the light source as it is in-coupled into the one or more waveguides. Note that in some embodiments, one or both of the display elements 120 are opaque and do not transmit light from a local area around the headset 100. The local area is the area surrounding the headset 100. For example, the local area may be a room that a user wearing the headset 100 is inside, or the user wearing the headset 100 may be outside and the local area is an outside area. In this context, the headset 100 generates VR content. Alternatively, in some embodiments, one or both of the display elements 120 are at least partially transparent, such that light from the local area may be combined with light from the one or more display elements to produce AR and/or MR content.

In some embodiments, a display element 120 does not generate image light, and instead is a lens that transmits light from the local area to the eyebox. For example, one or both of the display elements 120 may be a lens without correction (non-prescription) or a prescription lens (e.g., single vision, bifocal and trifocal, or progressive) to help correct for defects in a user's eyesight. In some embodiments, the display element 120 may be polarized and/or tinted to protect the user's eyes from the sun.

In some embodiments, the display element 120 may include an additional optics block (not shown). The optics block may include one or more optical elements (e.g., lens, Fresnel lens, etc.) that direct light from the display element 120 to the eyebox. The optics block may, e.g., correct for aberrations in some or all of the image content, magnify some or all of the image, or some combination thereof.

The DCA determines depth information for a portion of a local area surrounding the headset 100. The DCA includes one or more imaging devices 130 and a DCA controller (not shown in FIG. 1A), and may also include an illuminator 140. In some embodiments, the illuminator 140 illuminates a portion of the local area with light. The light may be, e.g., structured light (e.g., dot pattern, bars, etc.) in the infrared (IR), IR flash for time-of-flight, etc. In some embodiments, the one or more imaging devices 130 capture images of the portion of the local area that include the light from the illuminator 140. As illustrated, FIG. 1A shows a single illuminator 140 and two imaging devices 130. In alternate embodiments, there is no illuminator 140 and at least two imaging devices 130.

The DCA controller computes depth information for the portion of the local area using the captured images and one or more depth determination techniques. The depth determination technique may be, e.g., direct time-of-flight (ToF) depth sensing, indirect ToF depth sensing, structured light, passive stereo analysis, active stereo analysis (uses texture added to the scene by light from the illuminator 140), some other technique to determine depth of a scene, or some combination thereof.

The audio system provides audio content. The audio system includes a transducer array, a sensor array, one or more contact transducers 145. However, in other embodiments, the audio system may include different and/or additional components. Similarly, in some cases, functionality described with reference to the components of the audio system can be distributed among the components in a different manner than is described here. For example, some or all of the functions of the controller may be performed by a remote server.

The transducer array presents sound to user. The transducer array includes a plurality of transducers. A transducer may be a speaker 160 or a tissue transducer 170 (e.g., a bone conduction transducer or a cartilage conduction transducer). Although the speakers 160 are shown exterior to the frame 110, the speakers 160 may be enclosed in the frame 110. In some embodiments, instead of individual speakers for each ear, the headset 100 includes a speaker array comprising multiple speakers integrated into the frame 110 to improve directionality of presented audio content. The tissue transducer 170 couples to the head of the user and directly vibrates tissue (e.g., bone or cartilage) of the user to generate sound. The number and/or locations of transducers may be different from what is shown in FIG. 1A.

The sensor array (also referred to as a microphone array) detects sounds within the local area of the headset 100. The sensor array includes a plurality of acoustic sensors 180 and one or more contact transducers 145 (also referred to as contact microphones). An acoustic sensor 180 captures sounds emitted from one or more sound sources in the local area (e.g., a room). Each acoustic sensor is configured to detect sound and convert the detected sound into an electronic format (analog or digital). The acoustic sensors 180 may be acoustic wave sensors, microphones, sound transducers, or similar sensors that are suitable for detecting sounds.

In some embodiments, one or more acoustic sensors 180 may be placed in an ear canal of each ear (e.g., acting as binaural microphones). In some embodiments, the acoustic sensors 180 may be placed on an exterior surface of the headset 100, placed on an interior surface of the headset 100, separate from the headset 100 (e.g., part of some other device), or some combination thereof. The number and/or locations of acoustic sensors 180 may be different from what is shown in FIG. 1A. For example, the number of acoustic detection locations may be increased to increase the amount of audio information collected and the sensitivity and/or accuracy of the information.

The one or more contact transducers 145 detect tissue based vibrations resulting from speech of the user. A contact transducer 145 may be, e.g., a vibrometer, a contact microphone, an accelerometer, some other transducer that is configured to measure vibration through a surface, or some combination thereof. The one or more contact transducers 145 may be configured to be in contact with one or more portions of a head of the user. In the example shown in FIG. 1A, the contact transducer 145 is located in an area of the frame 110 that would be directly in contact with (the contact transducer 145 is directly touching the skin) and/or indirectly in contact (the contact transducer 145 is separated from the skin by one or more intermediate materials that transmit vibrations of the skin to the contact transducer 145) with a portion of a nose of a user wearing the headset 100. For example, it could be integrated into one or both nose pads of a set of glasses. In other embodiments, the contact transducer 145 may be located elsewhere on the headset 100 and/or there may be one or more additional contact transducers 145 on the headset 100 (e.g., could have one on each nose pad). When the user moves their jaw, sounds of the movement (and chewing) transmit through tissue of the user via tissue conduction. The sounds of movement (and chewing) manifest on the skin of the user's head as slight tissue based vibrations. The one or more contact transducers 145 detect these tissue based vibrations.

The controller 150 processes the detected tissue vibrations and information from the sensor array that describes sounds detected by the sensor array. The controller 150 may comprise a processor and a computer-readable storage medium. The controller 150 may be configured to generate direction of arrival (DOA) estimates, generate acoustic transfer functions (e.g., array transfer functions and/or head-related transfer functions), track the location of sound sources, form beams in the direction of sound sources, classify sound sources, generate sound filters for the speakers 160, train and/or use a signal processing and/or machine learning model, or some combination thereof.

The position sensor 190 generates one or more measurement signals in response to motion of the headset 100. The position sensor 190 may be located on a portion of the frame 110 of the headset 100. The position sensor 190 may include an inertial measurement unit (IMU). Examples of position sensor 190 include: one or more accelerometers, one or more gyroscopes, one or more magnetometers, another suitable type of sensor that detects motion, a type of sensor used for error correction of the IMU, or some combination thereof. The position sensor 190 may be located external to the IMU, internal to the IMU, or some combination thereof.

In some embodiments, the headset 100 may provide for simultaneous localization and mapping (SLAM) for a position of the headset 100 and updating of a model of the local area. For example, the headset 100 may include a passive camera assembly (PCA) that generates color image data. The PCA may include one or more RGB cameras that detect images of some or all of the local area. In some embodiments, some or all of the imaging devices 130 of the DCA may also function as the PCA. The images detected by the PCA and the depth information determined by the DCA may be used to determine parameters of the local area, generate a model of the local area, update a model of the local area, or some combination thereof. Furthermore, the position sensor 190 tracks the position (e.g., location and pose) of the headset 100 within the room.

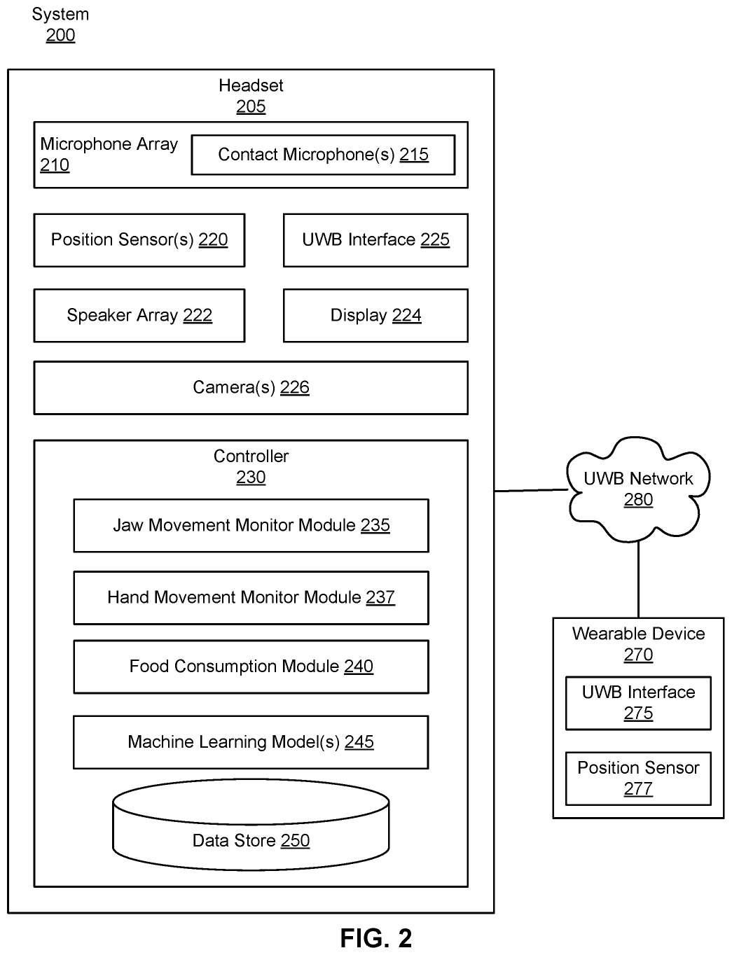

The controller 150 is an embodiment of the controller described below with regard to the UWB system, and may include some or all of the functionality of the controller of the UWB system. For example, the controller 150 may use the UWB communication channel to track the position of a hand (via the position of the wearable device) of the user relative to the head of the user. The controller 150 may monitor movement of a jaw of the user using a contact transducer 145. The controller 150 may determine a value of a food consumption parameter based in part on the tracked movement of hand and the monitored movement of the jaw. Additional details regarding the components of the headset 100 are discussed below in connection with FIG. 2.

FIG. 1B is a perspective view of a headset 105 implemented as an HMD, in accordance with one or more embodiments. In embodiments that describe an AR system and/or an MR system, portions of a front side of the HMD are at least partially transparent in the visible band (˜380 nm to 750 nm), and portions of the HMD that are between the front side of the HMD and an eye of the user are at least partially transparent (e.g., a partially transparent electronic display). The HMD includes a front rigid body 115 and a band 175. The headset 105 includes many of the same components described above with reference to FIG. 1A, but modified to integrate with the HMD form factor. For example, the HMD includes a display assembly, a DCA, an audio system, and a position sensor 190. FIG. 1B shows the illuminator 140, a plurality of the speakers 160, a plurality of the imaging devices 130, a plurality of acoustic sensors 180, a contact transducer 145, and the position sensor 190. The speakers 160 may be located in various locations, such as coupled to the band 175 (as shown), coupled to front rigid body 115, or may be configured to be inserted within the ear canal of a user. The contact transducer 145 may be also located in various locations, such as coupled to a side of the body 115 and/or the band 175 that is in contact with the head or face of the user.

FIG. 2 is a block diagram of a UWB system 200, in accordance with one or more embodiments. The system 200 includes a headset 205 and at least one wearable device 270. The headset 100 or 105 in FIG. 1A or FIG. 1B may be an embodiment of the headset 205. The wearable device 270 may be a watch, a bracelet, a ring, etc. Each of the headset 205 and the wearable device 270 includes a respective UWB interface 225 or 275 configured to communicate with each other via a UWB network 280.

UWB is a radio technology that uses low energy for short-range, high-bandwidth communications over a large portion of radio spectrum, e.g., >500 MHz (=million hertz). UWB transmissions transmit information by generating radio energy at specific time intervals and occupying a large bandwidth, thus enabling pulse-position or time modulation. The information can also be modulated on UWB signals (pulses) by encoding the polarity of the pulse, its amplitude and/or by using orthogonal pulses. UWB pulses can be sent sporadically at relatively low pulse rates to support time or position modulation, but can also be sent at rates up to the inverse of the UWB pulse bandwidth. This allows for the transmission of a large amount of signal energy without interfering with conventional narrowband and carrier wave transmission in the same frequency band. This also allows precise measurement of distances between two UWB devices.

In embodiments, the communication between the headset 205 and the wearable device 270 over the UWB network 280 can be used to identify a position of the wearable device 270 (i.e., a position of the hand of the user) relative to the head of the user.

The wearable device 270 includes a UWB interface 275. For example, the wearable device 270 may be a watch, a ring, and/or a wrist band, having a UWB interface. In some embodiments, the wearable device 270 also includes one or more position sensors 277. The UWB interface 275 is configured to transmit UWB signals to the headset 205. In some embodiments, the UWB interface 275 is configured to transmit short bursts of signals at a predetermined frequency. Based on the UWB communications between the wearable device 270 and the headset 205, the wearable device 270 or the headset 205 may identify their relative positions to each other.

In some embodiments, the one or more position sensors 277 are configured to measure the position of the wearable device 270. In some embodiments, the position sensors 277 may include an inertial measurement unit (IMU), accelerometers configured to measure the linear acceleration of the wearable device 270, and/or gyroscope sensors configured to measure the angular velocity of the wearable device 270. In some embodiments, responsive to detecting movement of the user's hand, the UWB interface 275 is caused to transmit signals to the headset 205. Alternatively, or in addition, responsive to detecting movement of the user's hand, the UWB interface 275 is caused to transmit signals to the headset 205 at a higher frequency.

The headset 205 includes a microphone array 210 that includes at least one contact microphone 215, one or more position sensors 220, and a controller 230. In some embodiments, the headset 205 may also include a speaker array 222, a display 224, and a camera 226. The microphone array 222 is configured to detect sounds from the local area. In some embodiments, the microphone array 222 includes one or more microphones and one or more contact microphones. A contact microphone is a type of microphone that is coupled to a skin of the user and measures the vibration of the skin. For example, a contact microphone may be an accelerometer, vibrometer, etc. In embodiments, the headset 205 includes at least one contact microphone 215. For example, the contact microphones may be part of a nose pad of the headset 205.

The one or more position sensors 220 are configured to measure a position of the headset 205. The measured position includes an orientation of the headset. In some embodiments, a position sensor is an inertial measurement unit (IMU), an accelerometer, and/or a gyroscope.

The one or more cameras 226 may be configured to monitor food objects and/or drink objects that have been consumed by the user. The display 224 is configured to display data generated by the UWB system 200.

The controller 230 is configured to receive data from the microphone array 210, position sensor(s) 220, speaker array 222, display 224, camera(s) 226, and/or the UWB interface 225, and process the received data to produce various output. The controller 230 includes a jaw movement monitor module 235, a hand movement monitor module 237, a food consumption module 240, and a data store 250. In some embodiments, the controller 230 also includes one or more machine learning models 245 and a data store 250.

The jaw movement monitor module 235 is configured to receive signals from the microphone array 210 and monitor the signals from the microphone array 210 to track movement of a jaw. In some embodiments, the jaw movement monitor model 235 is configured to process the signals from the microphone array 210 to identify sound patterns corresponding to different types of jaw movement or user actions. In some embodiments, the jaw movement monitor module 235 is configured to map different sound patterns to different user actions, such as (but not limited to) chewing, choking, drinking, speed of eating, talking, laughing, some other parameter describing an aspect of food consumption or jaw movement, or some combination thereof. For example, when the user is chewing food, the signals from the microphone array 210 may include a constant steady noise pattern generated by grinding food; when the user is choking, the signal from the microphone array 210 may indicate a burst sound; and when the user is talking, the signal from the microphone array 210 may include sound generated by moving of the jaw but not grinding.

In some embodiments, the patterns of jaw movement are identified by machine learning models. At least one machine learning model 245 may be trained using signals generated by contact microphones when the users are performing various jaw movements. The signals are labeled with different actions, such as chewing, choking, drinking, talking, laughing, etc. The machine learning model is trained to predict a user's action based on detecting the user's jaw movement.

The hand movement monitor module 237 is configured to receive signals from the wearable device 270, and monitor the signals from the wearable device 270 to identify and track the movement of the hand of the user relative to the headset 205. In some embodiments, the hand movement monitor module 237 is configured to analyze the signals received from the wearable device 270 to identify patterns. In some embodiments, these patterns may also correspond to different user actions, such as eating, choking, drinking, talking, laughing, etc. For example, when the user is eating, the user's hand often periodically moves back and forth between a plate and their mouth. As another example, when the user is talking, the user's hand may move in front of their body, but not too close to their mouth or head.

In some embodiments, the patterns of hand movement are also identified by machine learning. At least one machine learning model 245 may be trained using signals associated with hand movement of multiple users, labeled with different actions, such as chewing, choking, drinking, talking, laughing, etc. The machine learning model is trained to predict a user's action based on detecting the user's hand movement.

The food consumption module 240 is configured to further process the output of the jaw movement monitor module 235 and the hand movement monitor module 237 to determine values of one or more food consumption parameters. For example, when the jaw movement monitor module 235 detects a jaw movement corresponding to chewing, and the hand movement module 235 also detects a hand movement corresponding to eating, the food consumption model 240 may determine that the user is eating, and track an amount of time that the user is eating.

In some embodiments, the values of food consumption parameters are identified by machine learning. At least one machine learning model 245 may be trained using a first set of data associated with jaw movements of multiple users, and a second set of data associated with hand movements of multiple users, labeled with values of food consumption parameters. The machine learning module is trained to generate values of food consumption parameters based on detecting user's jaw movement and hand movement. A food consumption parameter may be, e.g., calories consumed, speed of eating, choking, food handling, some other parameter describing an aspect of food consumption, or some combination thereof.

In some embodiments, when food consumption module 240 finds that the user is choking, one or more of the devices (e.g., the headset 205, the wearable device 270, and/or a smart device) that are commutatively coupled to the system 200 may provide an alarm (such as an audible alarm, or a visible alarm) to bring the event to the attention of other people in the local area. In some embodiments, the alarm may be coupled to the headset 205. In some embodiments, the alarm may be coupled to the wearable device 270. In some embodiments, the alarm may be a stand-alone device placed in the environment of the user, or carried by a care giver of the user. In some embodiments, the system 200 may send a notification to a mobile device (e.g., a mobile phone) of the user, a care giver, and/or devices of medical personnel, or first responders.

In some embodiments, the machine learning model(s) 245 further includes one or more object recognition models trained to identify different food objects and/or drink objects based on images taken by the camera(s) 226. The machine learning model(s) 245 takes the images as input to determine whether there are food objects or drink objects in the images. In some embodiments, the machine learning model(s) 245 is trained to identify food objects or drink objects based on their packaging.

The data store 250 may be configured to store user data. The user may opt-in to allow the data store 250 to record data captured by the UWB system 200. In some embodiments, the UWB system 200 may employ always-on recording, in which the UWB system 200 records all signals captured by the UWB system 200 in order to improve the experience for the user. The user may opt in or opt out to allow or prevent the UWB system 200 from recording, storing, or transmitting the recorded data to other entities.

In some embodiments, the data store 250 further includes a portion configured to store calorie densities of a plurality of food objects or drink objects. In some embodiments, the controller 230 is further configured to retrieve a calorie density from the data store 250 based on the identified food objects or drink objects, and estimate a calorie consumption of the user based on the calorie density of the identified food object or drink object and other values of food consumption parameters, such as an amount of time eating, a number of times of chewing, speed of chewing, etc.

In some embodiment, the data store 250 also includes a portion configured to store other user data obtained from the headset 205, the wearable device 270, and/or other devices during a same period when the value of food consumption parameters are determined. Such data may include (but are not limited to) data associated with user's physical activities (such as physical exercise performed), or sleeping quality during the same period when the jaw movements and hand movements are tracked. In some embodiments, the controller 230 may also include additional modules configured to correlate the other data associated with the user with the values of the food consumption parameters of the user. The correlation can then be used to identify additional patterns, such as whether the values of the food consumption parameters are related to sleep qualities or amounts of exercise.

In some embodiments, the controller 230 is also configured to cause the analysis results, such as values of food consumption parameters generated by the food consumption module 240, jaw movement patterns identified by the jaw movement monitor module 235, and hand movement patterns identified by the hand movement monitor module 237, to be displayed on the display 224. In some embodiments, the user can also provide input on whether the analysis results of the controller 230 are correct or incorrect. The user input may then be taken as feedback, causing the controller 230 to update its algorithm and/or machine-learning models 245 to improve their future prediction results.

In some embodiments, the headset 205 and/or the wearable device 270 may also be communicatively coupled to another device, such as a smart phone (not shown). In some embodiments, some or all of the functionality of the controller 230 is performed by the smartphone.

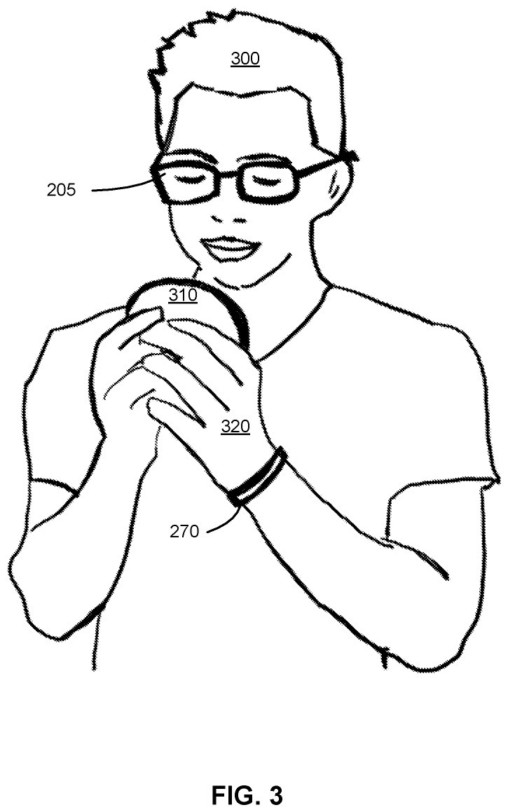

FIG. 3 illustrates an example use case of the UBE system 200. As illustrated, a user 300 is wearing the headset 205 and the wearable device 270. When the user 300 eats a hamburger, the jaw of the user 300 moves in a particular pattern, and the hand of the user 300 also moves in a particular pattern. For example, for each bite, the user moves his hand (that wears the wearable device 270 and holds the hamburger) closer to his mouth, takes a bite of the hamburger, starts chewing, and moves his hand away. The hand movement is detected by the position sensor 277 of the wearable device 270. The jaw movement (including biting and chewing) is detected by the contact microphone 215 coupled to the headset 205. The sensing data generated by the position sensor 277 is processed by the hand movement monitor module 237 to identify a pattern associated with eating finger food, and the sensing data generated by the contact microphone 215 is processed by the jaw movement monitor module 235 to identify a pattern associated with biting and chewing food.

The identified patterns, such as eating finger food, and biting and chewing food, are then processed by the food consumption module 240 to determine values of one or more food consumption parameters. For example, the eating finger food pattern identified by the hand movement monitor module 237 is compared with the biting and chewing pattern identified by the jaw movement monitor module 235 to determine that the user is likely eating finger food. The food consumption parameters may include a number of times chewing, a number of bites taken, etc.

In some embodiments, the headset 205 also includes a camera configured to take a picture of the hamburger with packaging showing the source of the hamburger (e.g., from a particular chain restaurant). The controller 230 of the headset 205 may then use machine learning models 245 to identify the source of the hamburger, retrieves a total calories of the hamburger from the data store 250, and records the total calorie of the hamburger as a value of a food consumption parameter.

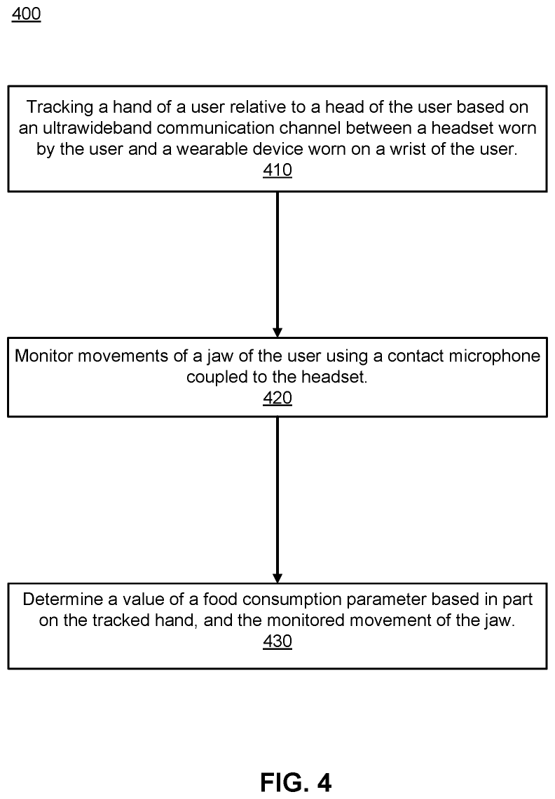

FIG. 4 is a flowchart of a method 400 for determining a value of a food consumption parameter, in accordance with one or more embodiments. The process shown in FIG. 4 may be performed by components of a UWB system (e.g., UWB system 200). Other entities (e.g., a server or a mobile device) may perform some or all of the steps in FIG. 4 in other embodiments. Embodiments may include different and/or additional steps, or perform the steps in different orders.

The UWB system tracks 410 a hand of a user (also referred to as “target user”) relative to a head of the user (e.g., user 300) based on signals transmitted via a UWB channel between a headset (e.g., headset 205) worn by the user and a wearable device (wearable device 270) worn on a wrist or a hand of the user. In some embodiments, a short burst of signals is transmitted between the wearable device and the headset periodically via the UWB channel. The distance between the headset and the wearable device can be measured precisely by measuring the time that it takes for a radio wave to pass between the two devices. In some embodiments, the wearable device includes a position sensor configured to detect the user's hand movement. In response to detecting the user's hand movement, the wearable device transmits a burst of signal to the headset via the UWB channel.

The UWB system monitors 420 movements of a jaw of the user using a contact microphone (e.g., contact microphone 215) coupled to the headset (e.g., headset 205). The contact microphone may be placed on a nose pad of the headset configured to be in contact with the skin of the user. The contact microphone is configured to detect skin vibration caused by jaw movements, such as chewing, talking, drinking, etc. The signal generated by the contact microphone may then be processed to identify a corresponding movement of the jaw or action of the user, such as chewing, talking, drinking, etc.

The UWB system determines 420 a value of a food consumption parameter based in part on the tracked movement of hand and the monitored movement of the jaw. For example, in some embodiments, determining the value of the food consumption parameter of the user includes accessing a machine-learning model trained on a dataset containing tracked movement of hands of users (which may or may not include the target user) relative to heads of the corresponding users, monitored jaw movements of the users, and values of the food consumption parameter of the users, and applies the machine-learning model to the tracked movement of the hand of the user relative to the head of the user, and the monitored movement of the jaw of the user to determine the value of the food consumption parameter of the user.

In some embodiments, determining 430 the value of the food consumption parameter of the user further includes identifying a pattern among a plurality of patterns of the jaw movement of the user. The plurality of patterns corresponds to at least one of chewing, drinking, or choking. In some embodiments, the method 400 further includes detecting choking of the user based on the identified pattern; and responsive to detecting choking of the user, sending an alert to another device.

In some embodiments, the method 400 further includes monitoring a food object or a drink object consumed by the user using a camera coupled to the headset. In some embodiments, monitoring the food object or the drink object further includes periodically taking images of objects that are within the user's reach; and identifying at least one of the images as the food object or the drink object using machine-learning models. In some embodiments, identifying the food object or the drink object is based on identifying the packaging of the food object or drink object.

In some embodiments, determining 430 the value of the food consumption parameter further includes retrieving a calorie density of the identified food object or drink object from a database; estimating a volume of the identified food object or the drink object that has been consumed based in part on the tracked movement of the hand and the monitored movement of the jaw; and determining a total calorie of the food object or drink object consumed based on the calorie density of the identified food object or drink object and the estimated volume of the identified food object or drink object.

In some embodiments, the method further includes correlating the value of the food consumption parameter with values of one or more second parameters associated with a second aspect of the user collected during a same time period. For example, in some embodiments, the one or more second parameters may be associated with an amount of exercise or hours of sleep of the user.

### Additional Configuration Information

The foregoing description of the embodiments has been presented for illustration; it is not intended to be exhaustive or to limit the patent rights to the precise forms disclosed. Persons skilled in the relevant art can appreciate that many modifications and variations are possible considering the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A method comprising:
tracking movement of a hand of a target user relative to a head of the target user based on an ultrawideband communication channel between a headset worn by the target user and a wearable device worn on the hand or a corresponding wrist of the target user;
monitoring movement of a jaw of the target user using a contact microphone coupled to the headset; and
determining a value of a food consumption parameter of the target user based in part on the tracked movement of the hand and the monitored movement of the jaw.

2. The method of claim 1, wherein determining the value of the food consumption parameter of the user comprises:
accessing a machine-learning model trained on a dataset containing (1) tracked movement of hands of users relative to heads of the corresponding users, (2) monitored jaw movements of the users, and (3) values of the food consumption parameter of the users; and
applying the tracked movement of the hand of the target user relative to the head of the user, and the monitored movement of the jaw of the target user to the machine-learning model to determine the value of the food consumption parameter of the target user.

3. The method of claim 2, wherein determining the value of the food consumption parameter of the user further comprises:
identifying a pattern among a plurality of patterns of jaw movements of the users, the plurality of patterns corresponding to at least one of chewing, drinking, or choking.

4. The method of claim 3, the method further comprising:
detecting choking of the target user based on the identified pattern; and
responsive to detecting choking of the target user, sending an alert to another device.

5. The method of claim 1, the method further comprising:
monitoring a food object or a drink object consumed by the user using a camera coupled to the headset.

6. The method of claim 5, wherein monitoring the food object or the drink object comprises:
periodically taking images of objects that are within reach of the target user; and
identifying at least one of the images as the food object or the drink object using machine-learning models.

7. The method of claim 5, wherein identifying the food object or the drink object is based on identifying packaging of the food object or the drink object.

8. The method of claim 5, wherein determining the value of the food consumption parameter comprises:
retrieving a calorie density of the identified food object or drink object from a database;
estimating a volume of the identified food object or the drink object that has been consumed based in part on the tracked movement of the hand and the monitored movement of the jaw of the target user; and
determining a total calorie of the food object or drink object consumed based on the calorie density of the identified food object or drink object and the estimated volume of the identified food object.

9. The method of claim 1, wherein determining the value of the food consumption parameter based in part on the tracked movement of the hand is performed by the headset.

10. The method of claim 1, the method further comprising:
accessing values of one or more second parameters associated with a second aspect of the target user collected during a same time period when the value of food consumption parameter is determined; and
correlating the value of the food consumption parameter with the values of the one or more second parameters associated of the target user.

11. The method of claim 10, wherein the one or more second parameters include at least a parameter associated with an amount of exercise or hours of sleep of the target user.

12. An ultrawideband (UWB) system comprising:
a headset configured to be worn on a head of a user comprising a contact microphone and a first UWB interface; and
a wearable device configured to be worn on a wrist or a hand of the user comprising, and a second UWB interface configured to communicate with the headset over a UWB communication channel,
wherein the headset is configured to:
track movement of a hand of the user relative to the head of the user based on the communication transmitted or received from the wearable device over the UWB communication channel;
monitor movement of a jaw of the user using the contact microphone; and
determine a value of a food consumption parameter of the user based in part on the tracked movement of the hand and the monitored movement of the jaw.

13. The UWB system of claim 12, wherein determining the value of the food consumption parameter of the user comprises:
accessing a machine-learning model trained on a dataset containing tracked movements of hands of users relative to heads of the corresponding users, monitored jaw movements of the users, and values of the food consumption parameter of the users; and
applying the machine-learning model to the tracked movement of the hand of the user relative to the head of the user, and the monitored movement of the jaw of the user to determine the value of the food consumption parameter of the user.

14. The UWB system of claim 13, wherein determining the value of the food consumption parameter of the user further comprises:
identifying a pattern among a plurality of patterns of the jaw movement of the user, the plurality of patterns corresponding to at least one of chewing, drinking, or choking.

15. The UWB system of claim 13, wherein the headset is further configured to:
detect choking of the user based on the identified pattern; and
responsive to detecting choking of the user, send an alert to another device.

16. The UWB system of claim 12, wherein the headset further comprises a camera configured to monitor a food object or a drink object consumed by the user.

17. The UWB system of claim 16, wherein monitoring the food object or the drink object comprises:
periodically taking images of objects that are within reach of the target user; and
identifying at least one of the images as the food object or the drink object using machine-learning models.

18. The UWB system of claim 16, wherein identifying the food object or the drink object is based on identifying packaging of the food object or the drink object.

19. The UWB system of claim 16, wherein determining the value of the food consumption parameter comprises:
retrieving a calorie density of the identified food object or drink object from a database;
estimating a volume of the identified food object or the drink object that has been consumed based in part on the tracked movement of the hand and the monitored movement of the jaw; and
determining a total calorie of the food object or drink object consumed based on the calorie density of the identified food object or drink object and the estimated volume of the identified food object.

20. The UWB system of claim 12, the headset is further configured to:
accessing values of one or more second parameters associated with a second aspect of the user collected during a same time period when the value of food consumption parameter is determined; and
correlating the value of the food consumption parameter with the values of one or more second parameters associated with a second aspect of the user.
